(19) United States
(12) Patent Application Publication
(10) Pub. No.: US 2016/0199001 A1
(43) Pub. Date: Jul. 14, 2016

(54) HEART RATE DETECTION EARPHONE

(71) Applicant: Cheng Uei Precision Industry Co., Ltd., New Taipei City (TW)

(72) Inventors: CHENG LEE, New Taipei City (TW); KUO YANG WU, New Taipei City (TW); WEN-BING HSU, New Taipei City (TW); HSIANG-LING CHUNG, New Taipei City (TW)

(21) Appl. No.: 14/594,125

(22) Filed: Jan. 10, 2015

(51) Int. Cl. A61B 5/00; A61B 5/024; H04R 1/10

(52) U.S. Cl. CPC A61B 5/6817; H04R 1/1091; A61B 5/02427

(57) ABSTRACT

A heart rate detection earphone is worn on an auricle which has a cavitas conchae. The heart rate detection earphone includes an earphone body matched with and buckled in the auricle, a light guiding module disposed to the earphone body, and an optical sensor. The light guiding module defines a sensing surface exposed out of the earphone body, and the sensing surface abuts against the cavitas conchae. The optical sensor includes a light emitter and a light receiver respectively coupled with the light guiding module. Light beams emitted by the light emitter are guided by the light guiding module to irradiate the cavitas conchae. The light beams emitted by the light emitter are reflected by the cavitas conchae, and then the reflected light beams reflected by the cavitas conchae are returned to and are received by the light receiver through the light guiding module.

### BACKGROUND OF THE INVENTION

1. Field of the Invention

The present invention generally relates to an earphone, and more particularly to a heart rate detection earphone.

2. The Related Art

At present, under the popularity of measurement devices, requirements of personal physiology monitoring and measurement of ambient statuses in markets are gradually improved. Such as in various fields of sports, physical trainings, alimentary control, routine monitoring, or physical therapies etc, if physiological parameters are continually monitored, it will assist people to evaluate effects more effectively. Volumes of most monitoring devices are larger, users are easily interfered when measuring the physiological parameters, and it is unbeneficial to continuously measure the physiological parameters. So a cost of measuring the physiological parameters is increased. In order to compensate for defects of the current measurement devices, an innovative device which is miniaturized, carried conveniently and can be used to continuously measure the physiological parameters is essential to be provided.

In order to achieve the effect of measuring the physiological parameters, except for miniaturizing the measurement devices, it's important to choose a measured position and a used sensor type. Different measured positions and used sensor types will significantly affect accuracies of measurement results.

Considering from the measured position measured by the measurement device, an ear is a better measured position. Firstly, the ear is close to a brain and a heart, so a renewed speed of blood can reflect various circumstances of a body well. Moreover, the ear is a relatively stable measured position, and an eyesight and movement of the measured person are hardly interfered when the measurement device is worn on the ear, so it's appropriate for the measurement device to continuously measure the physiological parameters. Thus, an earphone for measuring the physiological parameters, such as a heart rate detection earphone is emerged.

Currently, the general heart rate detection earphone mostly includes an earphone body which is matched with a shape of an auricle and is capable of being buckled in the auricle, a light emitter disposed in the earphone body, and a light receiver. When the heart rate detection earphone is worn on the ear, the light emitter faces to skin of an inner side of an ear canal of the auricle or emits light beams to skin of an inner side of a tragus of the auricle. After the light beams penetrate through the skin, the light beams are reflected by subcutaneous blood, strengths of the reflected light beams are changed with blood flow pulsation in vessels. So information of variations of heart rates, blood flow, etc can be interpreted by virtue of detecting signals of variations of the strengths of the reflected light beams, namely photoplethysmography (PPG) per hour.

However, the variations of the heart rates are measured by virtue of detecting the signals of the variations of the strengths of the reflected light beams per hour, so the measured position with intensive vessels is chosen to measure for improving accuracies of the signals of the variations of the strengths of the reflected light beams. Currently, the common heart rate detection earphones mostly proceed the measurement by virtue of penetrating through the skin of the inner side of the ear canal or the skin of the inner side of the tragus. The skin of the inner side of the ear canal or the skin of the inner side of the tragus is usually thinner and the measurement is easily interfered by environmental light sources that results in the unstable signals of the variations of the strengths of the reflected light beams.

### SUMMARY OF THE INVENTION

An object of the present invention is to provide a heart rate detection earphone worn on an auricle. The auricle has a cavitas conchae. The heart rate detection earphone includes an earphone body, a light guiding module and an optical sensor. The earphone body is matched with and buckled in the auricle. The light guiding module is disposed to the earphone body. The light guiding module defines a sensing surface exposed out of the earphone body, and the sensing surface abuts against the cavitas conchae. The optical sensor includes a light emitter and a light receiver. The light emitter and the light receiver are respectively coupled with the light guiding module. Light beams emitted by the light emitter are guided by the light guiding module to irradiate the cavitas conchae. The light beams emitted by the light emitter are reflected by the cavitas conchae, and then the reflected light beams reflected by the cavitas conchae are returned to and are received by the light receiver through the light guiding module.

As described above, the optical sensor of the heart rate detection earphone is capable of correctly projecting the light beams into the cavitas conchae by virtue of the sensing surface of the light guiding module being exposed out of the earphone body and abutting against the cavitas conchae, and light beams emitted by the light emitter of the optical sensor are reflected by the cavitas conchae, and then reflected light beams reflected by the cavitas conchae are returned to and are received by the light receiver of the optical sensor through the light guiding module, so that information of heart rates are measured through the cavitas conchae. Furthermore, the cavitas conchae has a relatively flat surface of skin, so it's easier for the optical sensor to abut against the surface of the skin for improving accuracies of the measurement, blood vessels of the cavitas conchae are intensive, so signals of variations of strengths of the reflected light beams are quite significant, and the skin of the cavitas conchae is thicker, so the measurement is hardly interfered by environmental light sources to get the stable signals of the variations of the strengths of the reflected light beams.

### BRIEF DESCRIPTION OF THE DRAWINGS

The present invention will be apparent to those skilled in the art by reading the following description, with reference to the attached drawings, in which:

FIG. 1 is a perspective view of the heart rate detection earphone in accordance with a first embodiment of the present invention;

FIG. 2 is an exploded view of the heart rate detection earphone of FIG. 1;

FIG. 3 is a schematic diagram of the heart rate detection earphone in accordance with a second embodiment of the present invention;

FIG. 4 is a schematic diagram of the heart rate detection earphone in accordance with a third embodiment of the present invention, wherein the heart rate detection earphone includes a fastening component;

FIG. 5 is a schematic diagram of the heart rate detection earphone of FIG. 4, wherein the fastening component is worn on an auricle;

FIG. 6 is a schematic diagram of the auricle of FIG. 5;

FIG. 7 is a schematic diagram of the heart rate detection earphone in accordance with a fourth embodiment of the present invention, wherein the heart rate detection earphone includes the fastening component;

FIG. 8 is a schematic diagram of the heart rate detection earphone of FIG. 7, wherein the fastening component is worn on the auricle;

FIG. 9 is a sectional view of the heart rate detection earphone of FIG. 1;

FIG. 10 is a sectional view of the heart rate detection earphone of FIG. 1, wherein optical paths are shown; and

FIG. 11 is a sectional view of the heart rate detection earphone in accordance with a fifth embodiment of the present invention.

### DETAILED DESCRIPTION OF THE PREFERRED EMBODIMENT

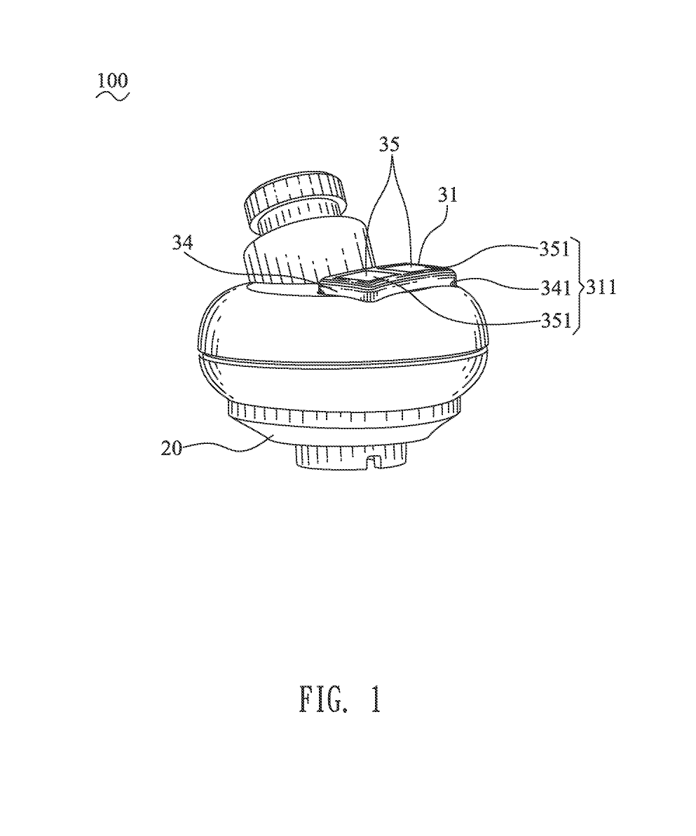

With reference to FIG. 1 to FIG. 11, a heart rate detection earphone 100 in accordance with the present invention is shown. The heart rate detection earphone 100 worn on an auricle 10, includes an earphone body 20 matched with and buckled in the auricle 10, a light guiding module 301 and an optical sensor 302. The optical sensor 302 includes a light emitter 32 and a light receiver 33. The auricle 10 has a cavitas conchae 18.

With reference to FIG. 1 to FIG. 11, the light guiding module 301 disposed to the earphone body 20, includes an opaque guiding base 34, and at least one nonopaque light guiding element 35 disposed to the guiding base 34. The light guiding module 301 defines a sensing surface 31. The sensing surface 31 is exposed out of the earphone body 20, and the sensing surface 31 abuts against the cavitas conchae 18. In the present invention, the light guiding module 301 includes two light guiding elements 35. Top surfaces of the guiding base 34 and the light guiding elements 35 are defined as the sensing surface 31.

With reference to FIG. 1 to FIG. 11, the heart rate detection earphone 100 detects a blood flow in blood vessels by the optical sensor 302. Information of the blood flow, heart rates and sympathetic responses are judged by virtue of different strengths of reflected light beams reflected by the red blood cells, other cells and tissues. Just because signals of variations of the strengths of the reflected light beams of the red blood cells are main detected signals, accuracies of the measured results are significantly affected by a measured position. If a density of the blood vessels of the measured position is higher, signals of the variations of the strengths of the reflected light beams are more significant.

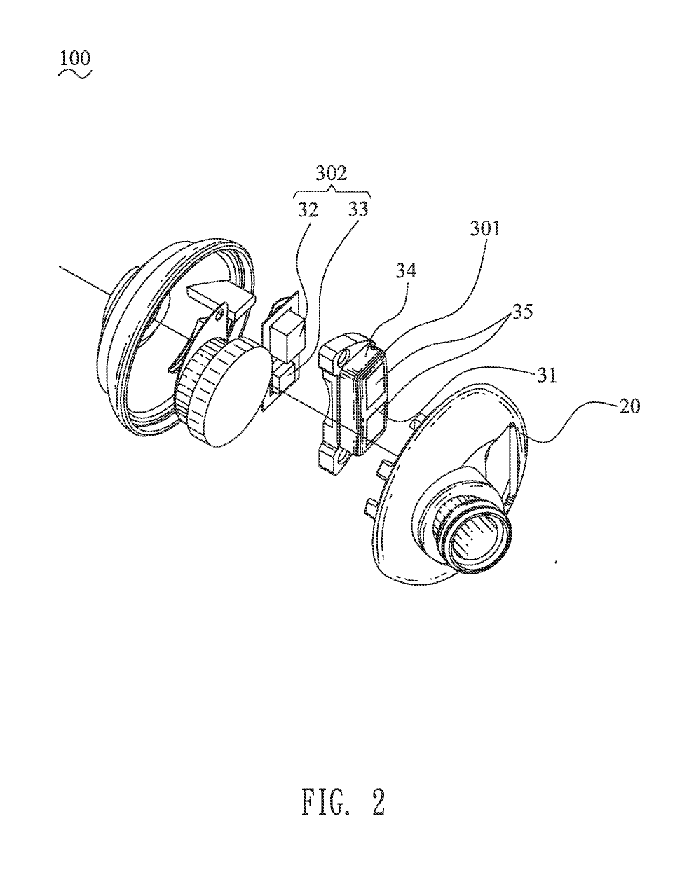

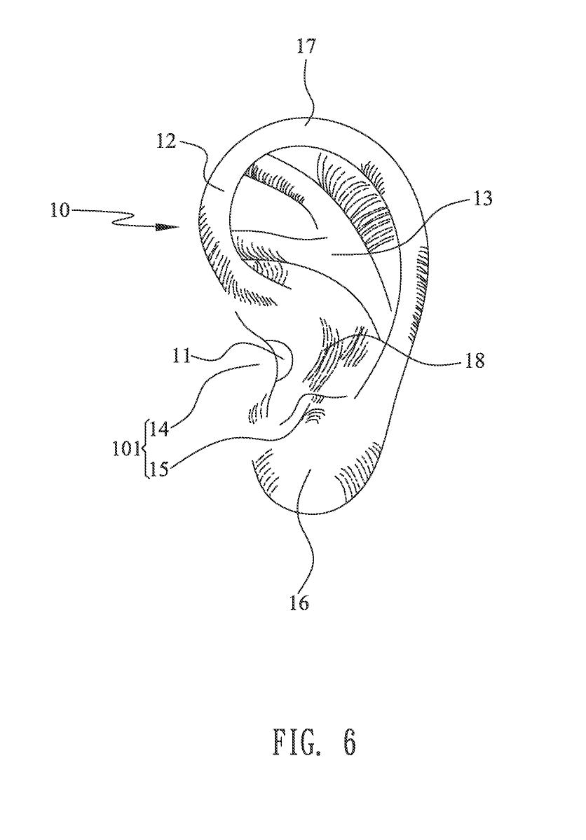

Referring to FIG. 2 and FIG. 6, the heart rate detection earphone 100 is worn on an auricle 10. The auricle 10 has an ear canal opening 11, two protruding structures 101 disposed around a periphery of the ear canal opening 11, and a helix 12 arched outward from the periphery of the ear canal opening 11 and spaced from the two protruding structures 101. The two protruding structures 101 are respectively a tragus 14 and an antitragus 15. The helix 12 forms an external contour of the auricle 10. A top of the helix 12 is designated as a tip of ear 17. A bottom of the helix 12 extends downward to form an auricular lobule 16.

Referring to FIG. 6, an apophysises and a wrinkle located between the ear canal opening 11 and the helix 12 form an antihelix 13. A relatively flat area located between the antihelix 13 and the antitragus 15 forms the cavitas conchae 18. The cavitas conchae 18 is the appropriate measured position for detecting the heart rates.

Referring to FIG. 2 and FIG. 6, reasons why the cavitas conchae 18 is the appropriate measured position for detecting the heart rates are described as follows. Firstly, comparing with other positions of the auricle 10, the cavitas conchae 18 has a relatively flat surface of skin, so it's easier for the optical sensor 302 to abut against the surface of the skin for improving the accuracies of the measurement. Secondly, the blood vessels of the cavitas conchae 18 are intensive, so the signals of the variations of the strengths of the reflected light beams are quite significant. Thirdly, the skin of the cavitas conchae 18 is thicker, so the measurement is hardly interfered by environmental light sources to get the stable signals of the variations of the strengths of the reflected light beams. Though the cavitas conchae 18 has various advantages of measuring the heart rates, it's difficult for the optical sensor 302 to be fastened on account of the flat structure of the cavitas conchae 18.

Referring to FIG. 2 and FIG. 6, in order to make the optical sensor 302 stably worn on the cavitas conchae 18, the optical sensor 302 is fastened in the heart rate detection earphone 100. The heart rate detection earphone 100 has broadcast, heart rate detection, etc functions. The heart rate detection earphone 100 includes an earphone body 20. The earphone body 20 is matched with the auricle 10, and is worn inside the auricle 10.

Referring to FIG. 2 and FIG. 6, the light emitter 32 and the light receiver 33 of the optical sensor 302 are respectively coupled with the light guiding module 301. Light beams emitted by the light emitter 32 of the optical sensor 302 are guided by the light guiding module 301 to irradiate the surface of the skin of the cavitas conchae 18. The light beams emitted by the light emitter 32 of the optical sensor 302 are reflected by the cavitas conchae 18, and then the reflected light beams reflected by the cavitas conchae 18 are returned to and are received by the light receiver 33 of the optical sensor 302 through the light guiding module 301.

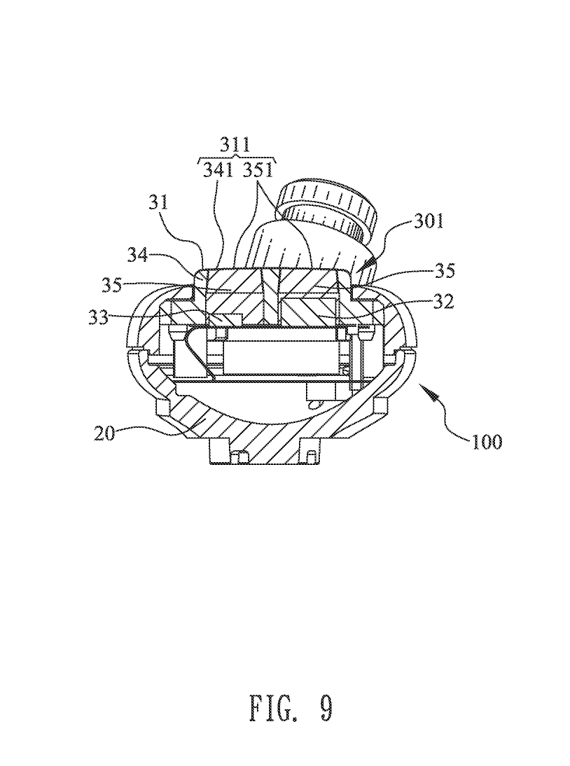

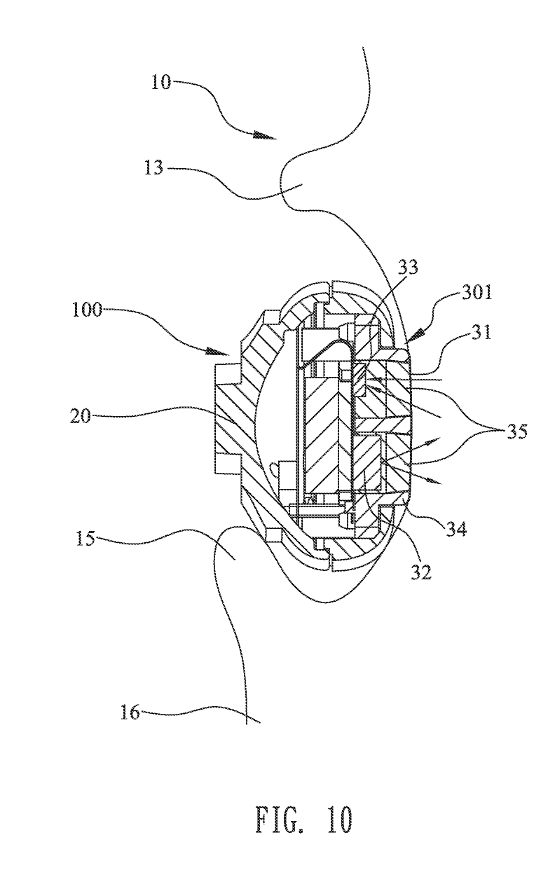

Referring to FIG. 1, FIG. 2, FIG. 6, FIG. 9 and FIG. 10, the heart rate detection earphone 100 in accordance with a first embodiment of the present invention is shown. In the first embodiment, the light emitter 32 and the light receiver 33 are disposed inside the earphone body 20. The light guiding module 301 further includes a light guiding element 35. The light emitter 32 and the light receiver 33 are without contacting the light guiding element 35 of the light guiding module 301 directly. The light guiding module 301 is disposed to optical paths of the light emitter 32 and the light receiver 33, thereby, the light beams emitted by the light emitter 32 are reflected by the cavitas conchae 18, and then the reflected light beams reflected by the cavitas conchae 18 are returned to and are received by the light receiver 33 through the light guiding module 301.

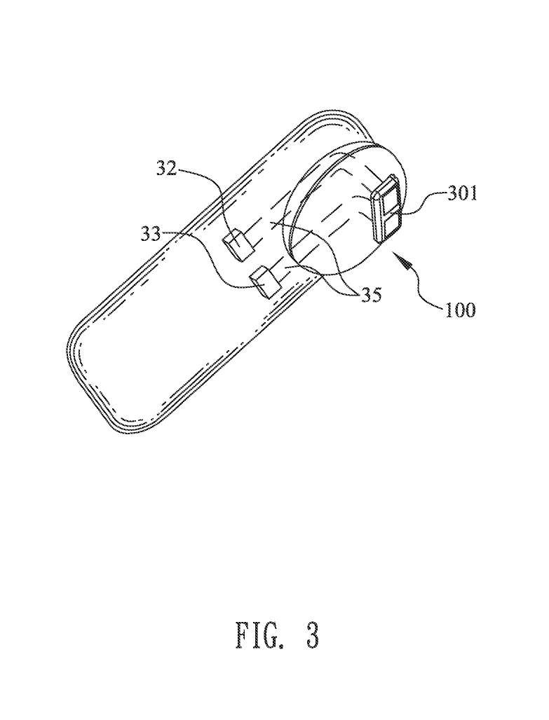

Referring to FIG. 3 and FIG. 6, the heart rate detection earphone 100 in accordance with a second embodiment of the present invention is shown. In the second embodiment, the light emitter 32 and the light receiver 33 are disposed outside the earphone body 20. The light emitter 32 and the light receiver 33 contact the light guiding element 35 of the light guiding module 301 directly. The light beams emitted by the light emitter 32 are guided by the light guiding module 301 to be gathered to a specific position of the cavitas conchae 18. The light beams emitted by the light emitter 32 are reflected by the cavitas conchae 18, and then the reflected light beams reflected by the cavitas conchae 18 are returned to and are received by the light receiver 33 through the light guiding module 301.

Referring to FIG. 2, FIG. 3, FIG. 6, FIG. 9 and FIG. 10, hence, the coupling described in the present invention is not limited to mechanically combine the light emitter 32 and the light receiver 33 with the light guiding module 301, but to dispose the light guiding module 301 to the optical paths of the light emitter 32 and the light receiver 33 for guiding the light beams emitted by the light emitter 32 or the reflected light beams reflected by the cavitas conchae 18.

Referring to FIG. 2, FIG. 3, FIG. 6, FIG. 9 and FIG. 10, the sensing surface 31 of the light guiding module 301 is exposed out of the surface of the earphone body 20 to abut against the surface of the skin of the cavitas conchae 18. The light guiding module 301 is disposed inside the earphone body 20. When the light guiding module 301 abuts against the cavitas conchae 18, an outer surface of the earphone body 20 abuts against the antitragus 15 of the auricle 10.

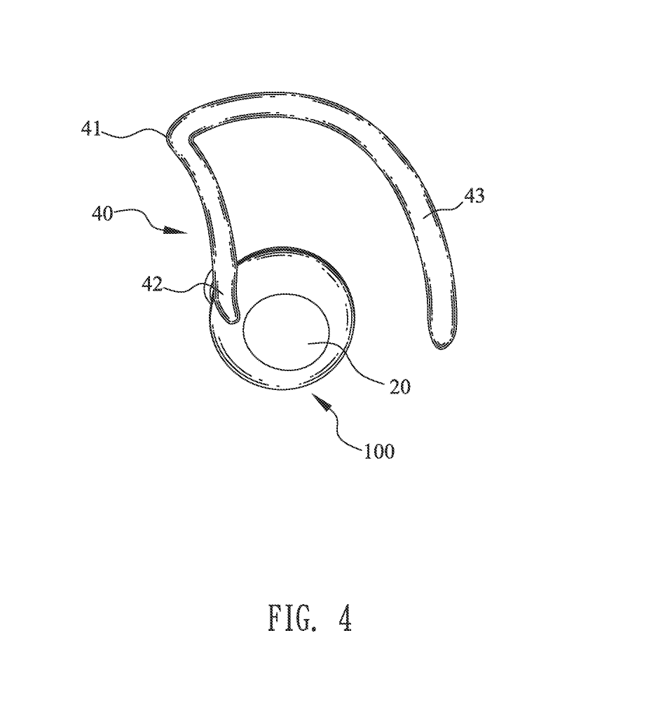

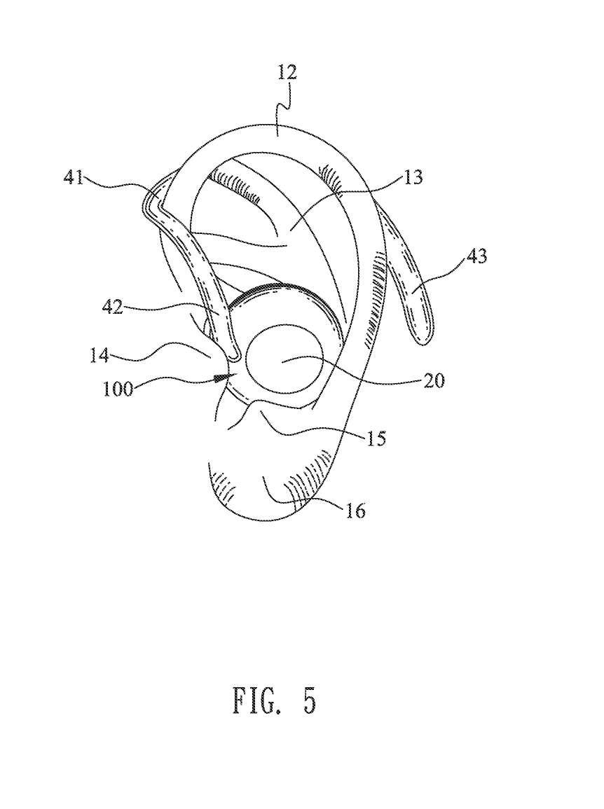

Referring to FIG. 4, FIG. 5 and FIG. 6, the heart rate detection earphone 100 in accordance with a third embodiment of the present invention is shown. In the third embodiment, the heart rate detection earphone 100 further includes a fastening component 40. In order to make the earphone body 20 stably worn on the auricle 10, the earphone body 20 is stably combined with the fastening component 40. The fastening component 40 is appropriate for a shape of the auricle 10 for improving a stability of wearing the earphone body 20. In the third embodiment, the fastening component 40 is an ear hook 41. One end of the ear hook 41 is defined as a first fastening portion 42 connected with the earphone body 20, and the other end of the ear hook 41 is defined as an elastic cantilever arm 43 matched with an outline of the helix 12 of the auricle 10. When the ear hook 41 is combined with the earphone body 20, the earphone body 20 is stably worn inside the auricle 10 by virtue of the cantilever arm 43 cooperating with the helix 12.

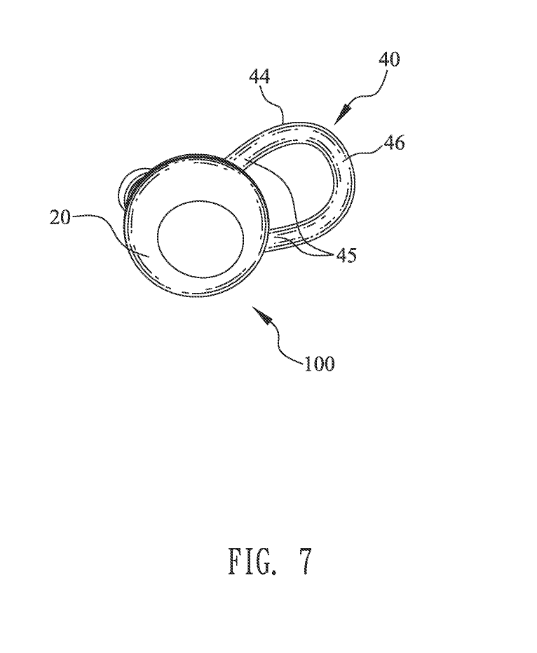

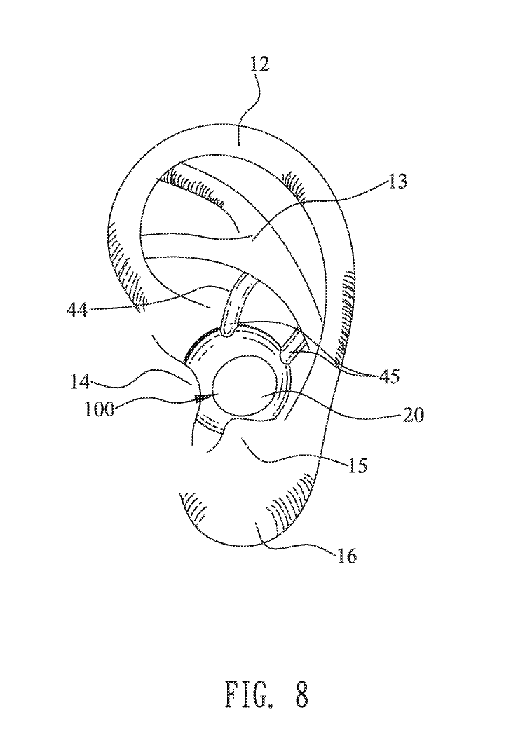

Referring to FIG. 6, FIG. 7 and FIG. 8, the heart rate detection earphone 100 in accordance with a fourth embodiment of the present invention is shown. In the fourth embodiment, the fastening component 40 is an earflap 44. One end of the earflap 44 is defined as a second fastening portion 45 connected with the helix 12 of the auricle 10. The other end of the earflap 44 is defined as an elastic buckling portion 46 matched with the antihelix 13 of the auricle 10. When the earflap 44 is combined with the earphone body 20, the earphone body 20 is stably worn inside the auricle 10 by virtue of the second fastening portion 45 cooperating with the antihelix 13.

Referring to FIG. 1 to FIG. 10, in the above-mentioned embodiments, in order to decrease affection of the environmental light sources, the top surfaces of the guiding base 34 and the light guiding elements 35 are smoothly connected for decreasing incident interferences of the environmental light sources. All parts of the top surfaces of the guiding base 34 and the light guiding elements 35 are successive, and surface curvatures of the light guiding elements 35 and the guiding base 34 are unobviously changed.

Referring to FIG. 1 to FIG. 10, the sensing surface 31 is defined as a spherical curved surface 311 protruded towards the cavitas conchae 18 to facilitate the sensing surface 31 abutting against the cavitas conchae 18. The spherical curved surface 311 includes a first spherical curved surface 341 and two second spherical curved surfaces 351. The top surface of the guiding base 34 is defined as the first spherical curved surface 341 protruded towards the cavitas conchae 18. The two top surfaces of the two light guiding elements 35 are defined as the two second spherical curved surfaces 351. Curvature radiuses of the first spherical curved surface 341 and the two second spherical curved surfaces 351 are the same so as to realize seamless connections among the guiding base 34 and the light guiding elements 35 and avoid causing surface curvatures of the sensing surface 31 to be changed.

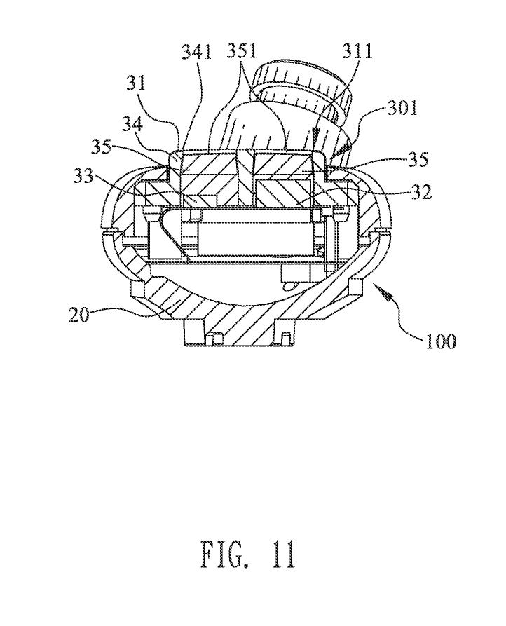

Referring to FIG. 11, the heart rate detection earphone 100 in accordance with a fifth embodiment of the present invention is shown. In the fifth embodiment, the top surface of the guiding base 34 is higher than the top surface of each of the light guiding elements 35 for decreasing incident interferences of the environmental light sources.

Referring to FIG. 1 to FIG. 11, the optical sensor 302 of the heart rate detection earphone 100 is capable of correctly projecting the light beams into the cavitas conchae 18 by virtue of the sensing surface 31 of the light guiding module 301 being exposed out of the earphone body 20 and abutting against the cavitas conchae 18, and the light beams emitted by the light emitter 32 of the optical sensor 302 are reflected by the cavitas conchae 18, and then the reflected light beams reflected by the cavitas conchae 18 are returned to and are received by the light receiver 33 of the optical sensor 302 through the light guiding module 301, so that the information of the heart rates are measured through the cavitas conchae 18.

Referring to FIG. 1 to FIG. 11, in addition, the light emitter 32 and the light receiver 33 can be designed as the light beams corresponding to various different wave lengths for cooperating physiological information which is to be measured. It's difficult for blue and violet light beams with shorter wave lengths to penetrate through the skin. The blue and violet light beams are appropriate to measure a relative movement between the earphone body 20 and the auricle 10 so as to judge and filter noises generated by the relative movement.

Referring to FIG. 1 to FIG. 11, it's easier for red and green light beams with longer wave lengths to project into blood vessels under the skin. So the red and green light beams are appropriate for measuring the blood flow. And infrared light can judge parameters of body temperatures. So the opaqueness and the nonopaqueness indicate whether the guiding base 34 and the light guiding elements 35 can be penetrated through by the light beams with the specific wave lengths which are to be used for measuring the physiological information. The specified light beams can be chosen to penetrate through the nonopaque light guiding element 35 and the unspecified environmental light beams are shielded for improving the accuracies of the measurement.

As described above, the optical sensor 302 of the heart rate detection earphone 100 is capable of correctly projecting the light beams into the cavitas conchae 18 by virtue of the sensing surface 31 of the light guiding module 301 being exposed out of the earphone body 20 and abutting against the cavitas conchae 18, and the light beams emitted by the light emitter 32 of the optical sensor 302 are reflected by the cavitas conchae 18, and then the reflected light beams reflected by the cavitas conchae 18 are returned to and are received by the light receiver 33 of the optical sensor 302 through the light guiding module 301, so that the information of the heart rates are measured through the cavitas conchae 18. Furthermore, the cavitas conchae 18 has the relatively flat surface of skin, so it's easier for the optical sensor 302 to abut against the surface of the skin for improving the accuracies of the measurement, the blood vessels of the cavitas conchae 18 are intensive, so the signals of the variations of the strengths of the reflected light beams are quite significant, and the skin of the cavitas conchae 18 is thicker, so the measurement is hardly interfered by the environmental light sources to get the stable signals of the variations of the strengths of the reflected light beams.

The forgoing description of the present invention has been presented for purposes of illustration and description. It is not intended to be exhaustive or to limit the invention to the precise form disclosed, and obviously many modifications and variations are possible in light of the above teaching. Such modifications and variations that may be apparent to those skilled in the art are intended to be included within the scope of this invention as defined by the accompanying claims.

What is claimed is:

1. A heart rate detection earphone worn on an auricle, the auricle having a cavitas conchae, the heart rate detection earphone comprising:
an earphone body matched with and buckled in the auricle;
a light guiding module disposed to the earphone body, the light guiding module defining a sensing surface exposed out of the earphone body, and the sensing surface abutting against the cavitas conchae; and
an optical sensor including a light emitter and a light receiver, the light emitter and the light receiver being respectively coupled with the light guiding module, light beams emitted by the light emitter being guided by the light guiding module to irradiate the cavitas conchae, the light beams emitted by the light emitter being reflected by the cavitas conchae, and then the reflected light beams reflected by the cavitas conchae being returned to and being received by the light receiver through the light guiding module.

2. The heart rate detection earphone as claimed in claim 1, wherein the light emitter and the light receiver are disposed inside the earphone body.

3. The heart rate detection earphone as claimed in claim 1, wherein the light guiding module is disposed inside the earphone body, when the light guiding module abuts against the cavitas conchae, an outer surface of the earphone body abuts against an antitragus of the auricle.

4. The heart rate detection earphone as claimed in claim 1, further comprising a fastening component, the earphone body being stably combined with the fastening component.

5. The heart rate detection earphone as claimed in claim 4, wherein the fastening component is an ear hook, one end of the ear hook is defined as a first fastening portion connected with the earphone body, and the other end of the ear hook is defined as an elastic cantilever arm matched with an outline of a helix of the auricle.

6. The heart rate detection earphone as claimed in claim 4, wherein the fastening component is an earflap, one end of the earflap is defined as a second fastening portion connected with a helix of the auricle, and the other end of the earflap is defined as an elastic buckling portion matched with an antihelix of the auricle.

7. The heart rate detection earphone as claimed in claim 1, wherein the light guiding module includes an opaque guiding base, and at least one nonopaque light guiding element disposed to the guiding base.

8. The heart rate detection earphone as claimed in claim 7, wherein the light guiding module includes two light guiding elements, top surfaces of the guiding base and the light guiding elements are defined as the sensing surface.

9. The heart rate detection earphone as claimed in claim 8, wherein the top surfaces of the guiding base and the light guiding elements are smoothly connected.

10. The heart rate detection earphone as claimed in claim 9, wherein all parts of the top surfaces of the guiding base and the light guiding elements are successive.

11. The heart rate detection earphone as claimed in claim 10, wherein the sensing surface is defined as a spherical curved surface protruded towards the cavitas conchae, the spherical curved surface includes a first spherical curved surface and two second spherical curved surfaces.

12. The heart rate detection earphone as claimed in claim 11, wherein the top surface of the guiding base is defined as the first spherical curved surface protruded towards the cavitas conchae, the two top surfaces of the two light guiding elements are defined as the two second spherical curved surfaces, curvature radiuses of the first spherical curved surface and the two second spherical curved surfaces are the same.

13. The heart rate detection earphone as claimed in claim 8, wherein the top surface of the guiding base is higher than the top surface of each of the light guiding elements.
